(19) United States
(12) Patent Application Publication
(10) Pub. No.: US 2009/0030265 A1
(43) Pub. Date: Jan. 29, 2009

(54) Surgical Instrument for Correcting Hypermotility of the Female Urethra in Exertional Urinary Incontinence

(76) Inventors: Donato Piroli Torelli, Napoli (IT); Mario Polichetti, Roccapiemonte (IT)

Correspondence Address: Modiano & Associati, Via Meravigli, 16, Milan, 20123, IT

(21) Appl. No.: 12/223,535

(22) PCT Filed: Nov. 2, 2006
(86) PCT No.: PCT/EP2006/010541

§ 371 Date: Aug. 1, 2008

(51) Int. Cl. A61B 17/00; A61F 2/00

(52) U.S. Cl. 600/29; 606/1

(57) ABSTRACT

A surgical instrument for correcting hypermotility of the female urethra in exertional urinary incontinence, comprising a rod-like body provided with a first rounded end and a second rounded opposite end provided with a slit.

The present invention relates to a surgical instrument for correcting hypermotility of the female urethra in exertional urinary incontinence. More particularly, the invention relates to a surgical instrument adapted to perform a surgical operation for correcting hypermotility of the female urethra so as to eliminate substantially the disorder of exertional urinary incontinence.

### BACKGROUND OF THE INVENTION

As is known, exertional urinary incontinence, i.e., the involuntary leakage of urine following exertion, such as for example coughing, lifting a weight, and in general whenever abdominal pressure increases for any reason, is a problem which can be corrected surgically.

One of the surgical procedures currently used consists in inserting, beneath the female middle urethra, a small polypropylene tape, which is passed through the obturator membrane of either side up to the skin (T.O.T.). The tape is approximately 1 cm thick and has several forms of weave.

The instrument currently used to perform the surgical procedure for positioning the tape consists of a channeling or tunneling tool, constituted by a helical needle with any curvature, which is mounted on a handpiece which allows to use it. Such devices can be of the right-handed and left-handed type, depending on the anatomical part to be crossed. One end of the helical needle is rounded and allows the engagement of the polypropylene tape in order to pass below the middle female urethra and is made to exit through the obturator membrane of both sides from the skin.

The instrument described above forces to perform the surgical procedure practically blindly, with the possibility of the onset of several severe complications for the patient. Since the procedure, as mentioned, is performed blindly, patients may suffer vascular arterial and venous complications, which moreover can be diagnosed late, in addition to bladder, vaginal and rectal nervous lesions with the possibility of fistulas.

### SUMMARY OF THE INVENTION

The aim of the present invention is to provide a surgical instrument for correcting hypermotility of the female urethra in exertional urinary incontinence which allows to minimize the risks linked to the surgical procedure performed by using said instrument.

Within this aim, an object of the present invention is to provide a surgical instrument for correcting hypermotility of the female urethra which allows to measure precisely the distance from the urethra to the ischiopubic ramus, in order to be able to cut the tape to the required size.

Another object of the present invention is to provide a surgical instrument which allows to prepare two bilateral vaginal tunnels in which the ends that are distal with respect to the urethra are narrower than the proximal ends.

Still another object of the present invention is to provide a surgical instrument which is highly reliable, relatively simple to provide and at competitive costs.

This aim and these and other objects, which will become better apparent hereinafter, are achieved by a surgical instrument for correcting hypermotility of the female urethra in exertional urinary incontinence, characterized in that it comprises a rod-like body provided with a first convex rounded end and a second concave rounded opposite end which is provided with a slit.

### BRIEF DESCRIPTION OF THE DRAWINGS

Further characteristics and advantages of the invention will become better apparent from the description of preferred but not exclusive embodiments of the surgical instrument according to the present invention, illustrated by way of non-limiting example in the accompanying drawings, wherein:

FIG. 1 is a perspective view of a first embodiment of the surgical instrument according to the present invention;

FIG. 2 is a perspective view of a second embodiment of the surgical instrument according to the present invention;

FIG. 3 is a view of a third embodiment of the surgical instrument according to the present invention;

FIG. 4 is a view of an embodiment of a tape which can be used with the surgical instrument according to the present invention.

### DESCRIPTION OF THE PREFERRED EMBODIMENTS

With reference to the Figures, the surgical instrument according to the present invention, generally designated by the reference numeral 1, comprises a rod-like body 1a, conveniently made for example of sterilizable plastic material, which has a first rounded convex end 2 and a second concave end 3 which is also rounded and provided with a slit 4 which allows the engagement of a mesh of the tape.

The convex end of the body of the surgical instrument has a dual function:

to measure precisely the distance from the urethra to the ischiopubic ramus, or to the preset point, in order to be able to cut a tape 10 to the required size;

to prepare two bilateral vaginal tunnels, in which the ends arranged distally with respect to the urethra are narrower than the proximal ends.

The concave end of the surgical instrument is instead designed to engage the tape in order to be able to insert it in the tunnels prepared previously and give the tape the required tension.

Conveniently, the body of the surgical instrument has a plurality of notches (starting from the convex end and mutually spaced for example by half a centimeter for a total of six centimeters) intended to indicate to the surgeon the size of the tape to be inserted. Such notches are conveniently applied on both sides of the instrument.

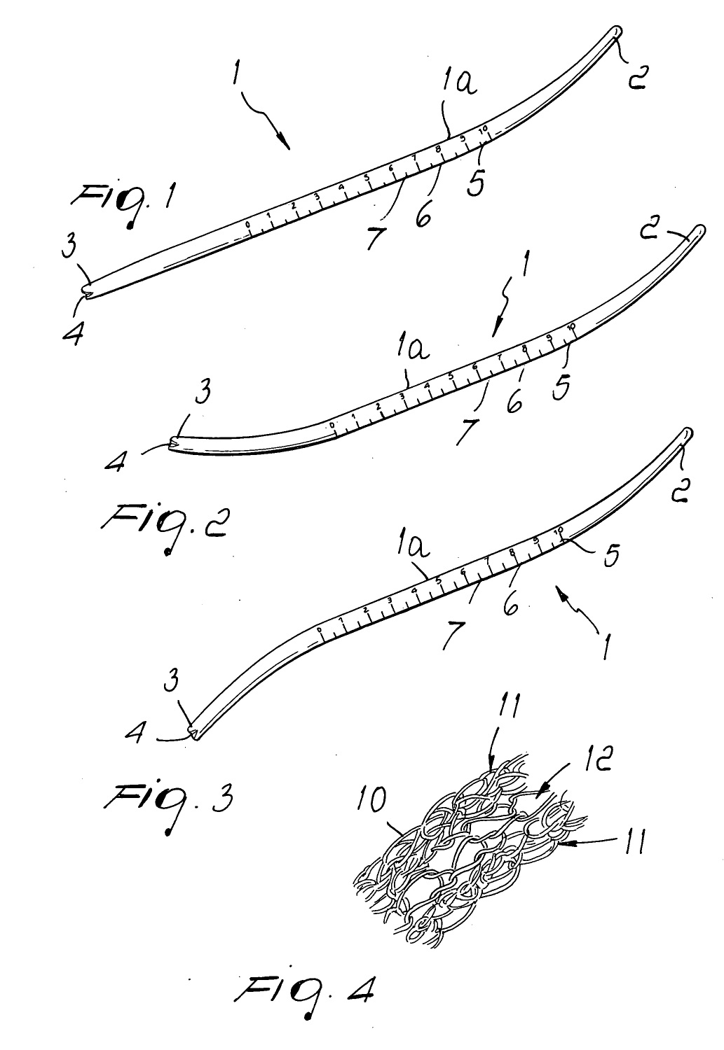

Conveniently, the surgical instrument can be provided by means of a body, as shown in FIG. 1, in which a convex end is curved with respect to the remaining portion of the body, or it can be as shown in FIG. 2, in which both ends, the convex one 2 and the concave one 3, are curved upwardly, or also as shown in FIG. 3, in which the body of the surgical instrument is S-shaped, with the convex end 2 and the concave end 3 curved respectively in opposite directions.

Moreover, the body of the surgical instrument 1 can be provided for example in straight form, not shown in the figures.

Conveniently, the surgical instrument has a diameter of for example 0.3 centimeters at its two ends and of 0.5 centimeters at the center, with a length ranging from 12 to 15 centimeters.

These measurements are of course merely an indication and may be changed according to the requirements.

The use of the surgical instrument according to the invention is as follows.

After disinfection of the area concerned, a linear incision of approximately 1 centimeter is produced starting approximately 1.5 centimeter below the urethral meatus. By means of the surgical instrument according to the invention, which is used only on the convex part, two channels are provided below the vaginal mucous membrane under the urethra until the two ischiopubic rami are touched bilaterally.

The tunnels thus formed are designed to accommodate a tape 10 with the weft for example as shown in FIG. 4, which is adapted to be engaged by the concave end of the surgical instrument and arranged transversely below the urethra so as to produce a supporting surface for the urethra proper.

At this point, by means of simple and completely nontraumatic maneuvers, the tape can be given the required tension after trimming it to size, by means of the graduated scale 5, 6, 7 arranged on the body of the surgical instrument. The small vaginal breach is then sutured and wadding is positioned and removed a few hours later.

Conveniently, the tape is provided with tight lateral meshes 11 and with relatively wider central meshes 12, so as to allow the engagement of the portion of the concave end 3 provided with the slot or slit 4 of the surgical instrument.

The surgical instrument thus conceived is susceptible of numerous modifications and variations, all of which are within the scope of the appended claims.

Thus, for example, the tape may be cylindrical in addition to the flat shape shown in FIG. 4.

All the details may further be replaced with other technically equivalent ones.

In practice, the materials used, as well as the contingent shapes and dimensions, may be any according to requirements and to the state of the art.

The disclosures in Italian Patent Application No. MI2006A000228 from which this application claims priority are incorporated herein by reference.

What is claimed is:

1-12. (canceled)

13. A surgical instrument for correcting hypermotility of the female urethra in exertional urinary incontinence, comprising a rod-like body provided with a first convex rounded end and a second concave rounded opposite end which is provided with a slit.

14. The surgical instrument according to claim 13, wherein said rod-like body is straight.

15. The surgical instrument according to claim 13, wherein said rod-like body has its convex end curved with respect to the rest of the body.

16. The surgical instrument according to claim 13, wherein said concave end and said convex end are curved with respect to the remaining portion of the rod-like body.

17. The surgical instrument according to claim 13, wherein said concave and convex ends are curved in opposite directions.

18. The surgical instrument according to claim 13, comprising, at the body of the instrument, a plurality of notches which are adapted to provide an indication of the measurement of a tape which must be cut to size by a surgeon.

19. The surgical instrument according to claim 18, wherein said notches are provided at least one side of the body of the instrument.

20. The surgical instrument according to claim 18, wherein said tape is substantially flat.

21. The surgical instrument according to claim 18, wherein said tape is substantially cylindrical.

22. A surgical kit for correcting hypermotility of the female urethra in exertional urinary incontinence, comprising:
two surgical instruments according to claim 13; and
a tape provided with lateral meshes which are tighter than central meshes.

23. The surgical kit according to claim 22, wherein said tape is substantially flat.

24. The surgical kit according to claim 22, wherein said tape is substantially cylindrical.
